(19) United States
(12) Patent Application Publication
(10) Pub. No.: US 2007/0190519 A1
(43) Pub. Date: Aug. 16, 2007

(54) Test

(76) Inventor: Shaun Kilminster, West Sussex (GB)

Correspondence Address: ELMORE PATENT LAW GROUP, PC, 209 MAIN STREET, N. CHELMSFORD, MA, 01863, US

(21) Appl. No.: 10/570,447

(22) PCT Filed: Sep. 6, 2004
(86) PCT No.: PCT/GB04/03803

§ 371 Date: Dec. 7, 2006

(30) Foreign Application Priority Data
Sep. 4, 2003 (GB) 0320728.9

(51) Int. Cl. C12Q 1/00

(52) U.S. Cl. 435/4

(57) ABSTRACT

The present invention relates to a method of measuring pain in an individual, by calculating the percentage ratio of one neurotransmitter to one or more other neurotransmitters, in a sample.

The present invention relates to a method for measuring pain in an individual, based on a sample, preferably a blood sample.

The measurement of pain is commonly used in the diagnosis of patients by doctors to give an indication of the severity of the injury, with patients often being asked to use a scale of 1-10 to define their level of pain. However, measurement of pain is subjective, varying from individual to individual. Pain measurement is also required to identify whether pain relief is effective. Small children, unconscious patients and animals are unable to communicate their level of pain, or provide further information as to the length of time that they have been suffering. An objective test for pain may also find use in the increasing number of claims against insurance policies.

Therefore there is a need for an objective test for pain.

Thus in the first aspect the present invention provides a method of measuring pain by calculating the percentage ratio of one neurotransmitter to one or more other neurotransmitters.

The method can further comprise the following steps:
(a) measuring the level of two or more neurotransmitters in a sample; and/or
(b) comparing the ratio with known ratios obtained from normal healthy controls and patients in pain.

In one preferred embodiment the neurotransmitters are selected from the group of Substance P (SP), 5-hydroxytryptamine (5HT) and Beta-endorphin (BE). The ratio of SP to 5HT and/or BE is preferably calculated.

The ratio of percentage SP/5HT and/or BE allows the diagnosis of pain to be determined, in this preferred embodiment.

For males a ratio of % SP/5HT of>90 indicates pain.

For females a ratio of % SP/5HT of>105 indicates pain.

For males a ratio of % SP/BE of>1150 indicates pain.

For females a ratio of % SP/BE of>1320 indicates pain.

In one preferred embodiment the sample is a blood sample.

The levels of SP and 5HT are preferably measured in the platelets. The level of BE is preferably measured in peripheral blood mononuclear cells (PBMC's).

In one preferred embodiment the pain is associated with headache or migraine. Other neurotransmitters may be useful for the diagnosis of pain associated with other symptoms or conditions.

Other transmitters that could be used include Neuropeptide-Y (N-Y), Vaso-active intestinal Peptide (VIP), and Calcitonin Gene Related peptide (CGRP) These neurotransmitters could be measured either in PBMC/platelets or plasma.

The method can be carried out on mammals, such as cats, dogs, horses, cattle, pigs, sheep or other domestic animals. Preferably the mammal is a human being.

The results of the test can be used to quantify the level of pain, into such categories as none, mild, moderate or severe. This can be evaluated by asking patients to answer a questionnaire, such as the Short Pain Inventory, Brief Pain Inventory; McGill Pain Questionnaire, or the Visual analogue rating at the time the sample is given. Other methods of subjectively measuring pain can also be used, and are well know to the person skilled in the art. By correlating the results of the test with the results of the questionnaires, the degree of pain can be quantified.

The present invention provides kits for use in the diagnosis of pain. Thus in a second aspect the present invention provides a kit for use in the diagnosis of pain comprising reagents for measuring the level of one or more neurotransmitters in a sample.

These kits may comprise one or more containers filled with one or more well known reagents for measuring the levels of neurotransmitters. These reagents may include antibodies, buffers, substrate mixtures, and instructions for their use. The antibodies may incorporate a label, which may be radioactive, fluorescent or a protein, such as, biotin or an enzyme, for example horse radish peroxidase. The substrate mixtures can be provided as individual components or as a ready prepared mixture.

The reagents may be provided in freeze-dried or lyophilised form or as a ready made solution. Such kits may also include other containers or devices for utilising the kit, and written instructions.

The present invention will now be described with reference to the following figures:

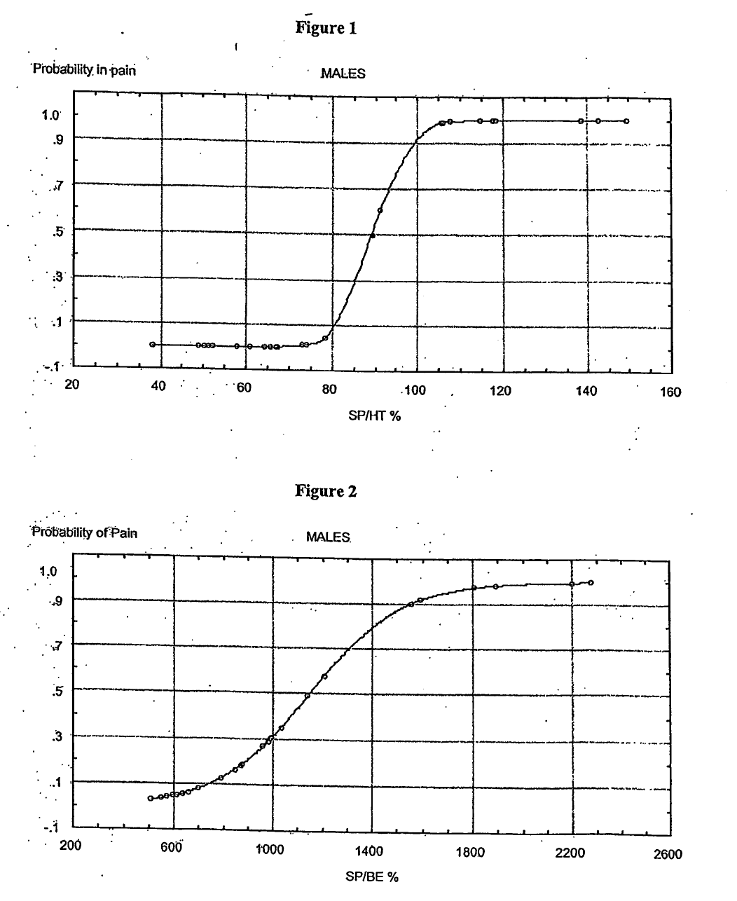

FIG. 1 shows the cumulative probability that a male is in pain as a function of SP/5HT % concentration.

FIG. 2 shows cumulative probability that a male is in pain as a function of SP/BE % concentration.

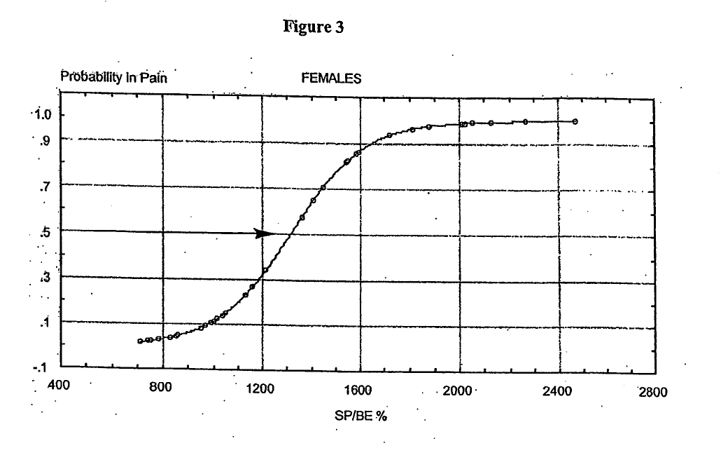

FIG. 3 shows cumulative probability that a female is in pain as a function of SP/BE % concentration.

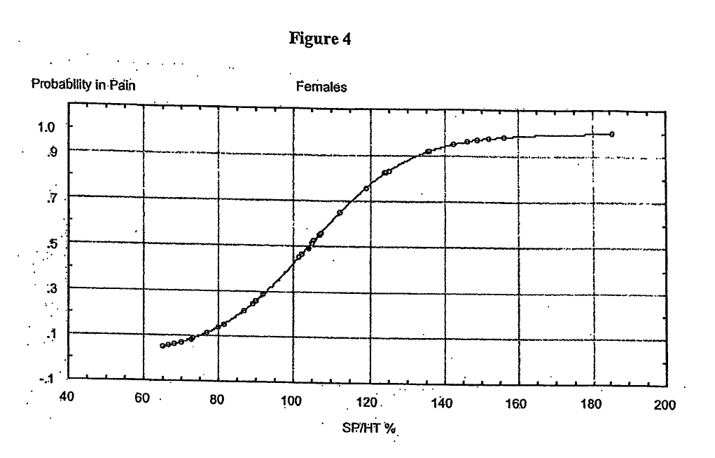

FIG. 4 shows cumulative probability that a female is in pain as a function of SP/5HT % concentration.

Any reliable cell separation method for mononuclear cells and platelets and any reliable method for detecting the intracellular amounts of the neurotransmitters (i.e. radio-immunoassay, HPLC (High Performance Liquid Chromatography)[refs. 1-5], Mass spectrometry [refs 6&7], LCMS (Liquid Chromatography-Mass Spectrometry) [refs 8-11]) could be used.

Details of methods suitable for measuring levels of substance P, 5-HT and beta-endorphin are given in the non-limiting examples below. However, other methods known to the skilled person can also be used.

### EXAMPLES

A sample of 30 (males+females) with pain for at least 24 hours and 30 age matched controls without pain for at least two weeks were recruited. Within 10 days of having pain, blood was drawn by simple venepunctures. Platelets and Peripheral blood mononuclear cells (PBMC) were separated by centrifugation and washing and then the cells were counted by any reliable method. These methods can be automated such as electrical impedance, light scatter/absorption, radio frequency energy, and laser light scatter or can involve the use of a microscope such as blood film examination or using a counting chamber [12, 13] Both control (non pain) and patients completed pain ratings to confirm pain or no pain status.

PBMC Harvesting:

8 ml venous blood samples were collected by venepunctures in heparinised (1000) UIL) tubes and PBMC by Ficoll-Hypaque gradient methods or similar. Platelet contamination was reduced to at least 0.3% by centrifuging the PBMC pellet three times at 100 g. Aprotinin (1000 KIU/mL) was added to the pellet to reduce the enzymatic degradation of proteins. Cells were counted. The cells were resuspended in 1 mL 0.1N acetic acid, homogenised in a blender, sonicated and then centrifuged at 20,000 g for 10 minutes. Supernatant was stored at −20 C until assay.

Platelets:

8 ml samples of venous blood samples were drawn into siliconised plastic tubes containing EDTA-2K or similar as the anticoagulant. Platelet rich plasma (PRP) was obtained by centrifugation at 300 g for 10 minutes at room temperature. The platelet rich pellet was separated from platelet-poor plasma by centrifuging PRP at 7000 g for 10 minutes. The platelet pellet was gently washed twice with HEPES-Tyroid buffer (145 mM NaCl, 5 mM KCl, 1 mM MgSO4, 10 mM N-2-hydroxyethylpiperazineN′-2-ethan sulphonic acid (HEPES) and 10 mM glucose per litre (pH7.4). The platelets were counted. All platelets were resuspended at the concentration of 1×10⁹ per mL and frozen at−80° C. until assay.

Estimation of Substance P in Platelets:

Frozen platelets were thawed and resuspended in 0.1N acetic acid, homogenised, sonicated and centrifuged at 20,000 g for 10 minutes. Substance P was eluted from the mixture with 60% acetonitrile in 0.1% trfluoroacetic acid (TFA), in a SEP column C18 with 0.1% TFA and 60% acetonitrile in 0.1% TFA. The eluate was evaporated to dryness in a centrifugal concentrator (Supervap PL-CC-180). To construct standard curves a serial fourfold dilution series from 3 pg/ml to 12 ng/ml of SP in RIA buffer was produced. The residue was dissolved in buffer for radio immunoassay. The determination of substance P in platelet and standard sample was performed with RIA kits (Peninsula Labs, Belmont Calif. USA. data were expressed as pg/10⁹ platelets. Cross reactivities with endothalin 1, neurokinin A and neuropeptide K are<0.05%, <0.01% and 0.01% respectively.

Estimation of Beta-endorphin in PBMC:

Serial dilutions of B-endorphins were prepared to construct standard curves. Beta-endorphin immunoreactivity was determined by means of radio-immuno assay (RIA) kits from Peninsular Labs, (or similar), Belmont USA. Data were expressed as pg/10⁶ cells.

Estimation of 5HT in Platelets:

All platelets are that were re suspended at the concentration of 1×1×10⁹ per mL and frozen at −80 until assay are thawed at room temperature and sonicated just prior to 5HT determination. Platelet 5HT is measured using reverse phase high performance liquid chromatography (HPLC) with electrochemical detection.

Platelet levels of 5HT were expressed as ng/10⁹ platelets. Any other relevant analytical method can be used e.g. MS or LCMS.

Results:

General Statistical Method.

It was found that even when patients and controls (non pain) were age matched, there were sex differences between the means of each neurotransmitter. The discriminant function analysis was carried out in Statistica Software but any other comparable statistical package would do the same eg SAS or SPSS.

The three neurotransmitters allow various combinations to be computed but the simple ratios of two neurotransmitters fed into a regression or discriminant function equation we have found to be effective. The simple t-tests statistics are shown in table 1-2. We computed t-tests for differences between pain or no pain groups. The use of discriminant function analysis allowed a more elegant understanding of the relationship between each pair or neurotransmitters and the probability that a subject had experienced pain or not.

The method can be replicated for any sub-samples i.e. children, race, concomitant drug treatment. The method may be replicated for a higher animal e.g. dog, horse rat.

Student t-test for Independent Samples Pain(Patient) v No Pain(controls) MALES

| | | 2-Tailed | | | Cont | Pat. | Cont | Pat | | |
|---|---|---|---|---|---|---|---|---|---|---|
| Dep. Var. | DFP % | t | df | p | N | N | Mean | Mean | Std. Dev. | Std. Dev. |
| BE | 64 | 3.377385 | 23 | 0.0025969 | 15 | 10 | 38.26 | 29.43 | 5.62657 | 7.454015 |
| HT5 | 96 | 7.767338 | 23 | 0.0000001 | 15 | 10 | 481.3333 | 350.05 | 43.61192 | 37.70556 |
| SP | 84 | −5.81088 | 23 | 0.0000064 | 15 | 10 | 286.4533 | 415.21 | 43.41204 | 67.79833 |
| SP/BE % | 80 | −4.93962 | 23 | 0.0000542 | 15 | 10 | 775.0059 | 1520.865 | 213.2357 | 528.0745 |
| SP/HT % | 100 | −9.91494 | 23 | 0 | 15 | 10 | 60.1405 | 119.1486 | 11.16526 | 18.68635 |

Student t-test for Independent Samples Pain(Patient) v No Pain(controls) FEMALES

| Dep. Var. | DFP % | t | df | p | N | N | Mean | Mean | Std. Dev. | Std. Dev. |
|---|---|---|---|---|---|---|---|---|---|---|
| BE | 82.85 | 4.935303 | 33 | 0.0000223 | 15 | 20 | 35.46 | 27.665 | 3.99031 | 5.040392 |
| HT5 | 71.42 | 2.133331 | 33 | 0.0404232 | 15 | 20 | 407.6533 | 366.98 | 68.32942 | 44.39862 |
| SP | 88.57 | −5.96416 | 33 | 0.0000011 | 15 | 20 | 328.9667 | 450.44 | 31.05303 | 73.92584 |
| SP/BE % | 91.42 | −6.4995 | 33 | 0.0000002 | 15 | 20 | 941.2274 | 1685.264 | 154.6709 | 421.2668 |
| SP/HT % | 82.86 | −5.26169 | 33 | 0.0000085 | 15 | 20 | 82.84868 | 125.0357 | 16.15076 | 27.65532 |

The dependent variable was entered as Pain=1 or No pain 0 as a dummy variable. This variable was regressed upon the % scores of each neurotransmitter. The discriminant function analysis output gave the posterior probabilities for each case with correct/incorrect classifications. These probabilities were FIGS. 1-4 shows the cumulative probability that a female is in pain as a function of % ratio concentrations. The classification results were as follows. A regression equation allows any value of % neurotransmitters to be entered into the equation and the probability that a subjects is in pain can be computed on an individual basis.

MALES ONLY: Discriminant function
Model with ratio scores SP/5HT %

| | | | |
|---|---|---|---|
| | Percent | CONT | PATIENT |
| Group | Correct | p = .50000 | p = .50000 |

Classification Matrix:
DISCRIM. Rows: Observed classifications
STATS Columns: Predicted classifications

| | | | |
|---|---|---|---|
| CONT | 100. | 15 | 0 |
| PATIENT | 100. | 0 | 10 |
| Total | 100. | 15 | 10 |

Now second method Ratio in males SP/BE %
Classification Matrix
Rows: Observed classifications
Columns: Predicted classifications

| | | | |
|---|---|---|---|
| Group | Correct | p = .50000 | p = .50000 |
| CONT | 93.3 | 14 | 1 |
| PATIENT | 60.0 | 4 | 6 |
| Total | 80.0 | 18 | 7 |

FEMALE Discriminant function Model CALCULATION
OF PAIN STATUS FROM SP/BE %

| | | | |
|---|---|---|---|
| | Percent | CONT | PATIENT |
| Group | Correct | p = .50000 | p = .50000 |

Classification Matrix
Rows: Observed classifications
Columns: Predicted classifications

| | | | |
|---|---|---|---|
| CONT | 100. | 15 | 0 |
| PATIENT | 85. | 3 | 17 |
| Total | 91.43 | 18 | 17 |

Now a different Ratio in females discriminant function SP/5HT:
Classification Matrix
Rows: Observed classifications
Columns: Predicted classifications

| | | | |
|---|---|---|---|
| | Percent | CONTROL | PATIENTS |
| Group | Correct | p = .50000 | p = .50000 |
| CONT | 86.66666 | 13 | 2 |
| PATIENT | 80.00000 | 4 | 16 |
| Total | 82.85714 | 17 | 18 |

The method has been shown to work effectively in both males and females with 100% correct prediction accuracy in males and 91% correct prediction in females. The method involves the calculation of substance P (SP), Beta..Endorphin and 5-hydroxytryptamine (5HT) in cells harvested from a sample of patients in pain and demographically matched controls who have not experienced pain for at least 1 month or greater. If patients have a migraine or tension type headache, then they go through a period of at least 7 days non treatment after the episode when the markers are collected by simple venepunctures. (The blood may be harvested on the same day as the pain, but the data gathered here were obtained in drug free patients).

The method may be refined by using discriminant function or regression techniques to put a value on a) the probability that a person was in pain and b) the degree of pain the subject was in. The outcome variable would be patient and matched non-pain controls status for method a). The outcome variable would be any validated subjective test (Short Pain Inventory SPI, McGill Pain Questionnaire MPQ, etc) of subjective pain over the last 24 hours or more for patient and matched non-pain controls for method b). Population data taking concomitant drug treatments may also be specified with the same methods. The method was shown to be effective in headache and is likely to be valid in other painful conditions. It is likely to work in children and possibly in higher animals.

References:

1. www.phann.uky.edu/ASRG/HPLC./hplcmytry.html
2. Beckman Model 330 HPLC Manuel, Beckman Instruments, Fullerton, Calif.
3. Knox, J. H. and Kauer, B.; High Performance Liquid Chromatography; Brown. P. R. and Hartwick, R. A. Eds.; Wiley Interscience: New York, 1989, Chapter 4.
4. Mant, C. T. and Hodges R. S. eds. High-Performance Liquid Chromatography of Peptides and Proteins: Separations, Analysis, and Conformation, CRC Press: Boston, 1991.
5. McClure, W. F. Analytical Chemistry, 1994, Vol. 66, pp. 43-54.
6. www.spectroscopynow.com/Spy/basehtml/SpyH/
7. Handbook of Molecular Physics and Quantum Chemistry. Stephen Wilson (Editor), Roy McWeeny (Editor), Peter Bemath (Editor). ISBN: 0-471-62374-1. Published by John Wiley.
8. www.1cms.com/
9. http://i-mass.com/
10. www.ionsource.com/
11. A Global View of LC/MS, How to Solve Your Most Challenging Analytical Problems. Ross Willoughby, Ed Sheehan & Sam Mitrovich (2nd. edition, 2002) ISBN 0-9660813-0-7.
12. Liquid Chromatography-Mass Spectrometry. W. M. A. Niessen (1998, 2nd edition) ISBN 0-8247193-6-0.
13. Hematology: Basic Principles and Practice. Edited by Ronald Hoffinan, Edward J. Benz, Jr, Sanford J. Shattil, Bruce Furie, Harvey J. Cohen, Leslie E. Silberstein, and Philip McGlave, 3rd ed, 2648 pp, ISBN 0-443-07954-4, New York, N.Y., Churchill Livingstone, 2000.
14. James H. H. Jandl. Blood: Textbook of Hematology. ISBN: 0316457310.(1996). Publisher: Lippincott Williams & Wilkins

1. A method of measuring pain by calculating the percentage ratio of one neurotransmitters to one or more other neurotransmitters.

2. A method as claimed in claim 1 further comprising the following steps:
(c) measuring the level of two or more neurotransmitters in a sample; and/or
(a) comparing the ratio with known ratios obtained from normal healthy controls and patients in pain.

3. A method as claimed in claim 1 or claim 2 wherein the neurotransmitters are selected from the group consisting of substance P, 5-hydroxytryptamine and Beta-endorphin.

4. A method as claimed in claim 2 or claim 3 wherein the sample is a blood sample.

5. A method as claimed claim 4 wherein the level of substance P is measured in platelets.

6. A method as claimed in claim 4 wherein the level of 5-Hydroxytryptamine is measured in platelets.

7. A method as claimed in claim 4 wherein the Beta-Endorphin is measured in peripheral blood mononuclear cells (PBMC's).

8. A method as claimed in any one of claims 1 to 7 wherein the percentage ratio calculated is that of substance P to 5-hydroxytryptamine and/or substance P to Beta-endorphin.

9. A method as claimed in any one of claims 1 to 8 comprising the following steps:
(a) measuring the level of substance P in a sample;
(b) measuring the level of 5-hydroxytryptamine (5HT) and/or Beta endorphin in said sample; and
(c) calculating the % ratio of Substance P to 5-hydroxytryptamine (5HT) and/or Beta endorphin (BE); and
(d) comparing the ratio with known ratios obtained from normal healthy controls and patients in pain.

10. A method as claimed in any one of claims 1 to 9 wherein a ratio of % SP/5HT of>90 in males and a ratio of % SP/5HT of>105 in females indicates pain.

11. A method as claimed in any one of claims 1 to 10 wherein a ratio of % SP/BE of>1150 in males and/or a ratio of % SP/BE of>1320 in females indicates pain.

12. A method as claimed in any one of claims 1 to 11 wherein the pain is caused by headache and or migraine.

13. A kit for use in the diagnosis of pain comprising reagents for measuring the level of one or more neurotransmitters in a sample.
